(19) United States
(12) Patent Application Publication
(10) Pub. No.: US 2008/0014225 A1
(43) Pub. Date: Jan. 17, 2008

(54) Method and system of manufacturing master-batch of ultra fine talc powders

(76) Inventors: Yun Fang Wu, Nin Shen City (CN); Yong Feng Liu, Liao Yang City (CN)

Correspondence Address: Raymond Y. Chan, Suite 128, 108 N. Ynez Avenue, Monterey Park, CA, 91754, US

(21) Appl. No.: 11/486,961

(22) Filed: Jul. 13, 2006

(51) Int. Cl. A61K 8/26

(52) U.S. Cl. 424/401

(57) ABSTRACT

A method of manufacturing ultra-fine talc powder materials includes the steps of (a) adding a predetermined amount of pre-treatment agent with a predetermined amount of talc powders to prevent the talc powders from agglomerating with each other; (b) performing activating treatment to the pre-treated talc powders to form activated talc powders for enhancing a compatibility of the talc powders with a predetermined plastic materials; (c) mixing the activated talc powders with a predetermined proportion of fluidized plastic materials to form a fluidized plastic-powders mixture, wherein the fluidized plastic-powder mixture is condensed to form solid plastic-powders mixture; and (d) hot-cutting the solid plastic-powders mixture until the solid plastic-powders mixture forms the ultra-fine talc powders materials as raw materials for manufacturing plastic products.

### BACKGROUND OF THE PRESENT INVENTION

1. Field of Invention

The present invention relates to powders, and more particularly to a system and method of manufacturing a master-batch of talc powders which are ultra fine in diameter and optimal for use in materials processing.

2. Description of Related Arts

Nowadays, powder technology has become one of the most important areas in manufacturing technology. There are particular braches of study for powder technology, viz. design and manufacturing of powders (e.g. manufacturing of ultra-fine powders materials and nano-powders materials), and pre-treatment of powders so that they can be readily mixed with other raw materials for performing advanced manufacturing process.

Traditionally, with regards to the first particular branch of powder technology, the core problem for engineers is to resolve the problem of powder agglomeration so as to ensure that each powder particle will effectively perform its respective role in a specific manufacturing process. More specifically, there exists a deep-seated problem for manufacturing of ultra-fine talc powders which are to be used in manufacturing plastic products. In particular, the smaller the talc powders, the more severe the problem of powder agglomeration, and the more severe the problem of powder agglomeration, the more problematic when they are mixed with raw plastic materials for manufacture of plastic products. Apart from all these, there exists another problem concerning flowability of powders. Powders may behave satisfactorily one day under a specific pressure, temperature and humidity, but flow poorly the next. Sometimes, small differences in moisture content, particle size, storage time, and even temperature can make a big difference in flowability. Thus, it is necessary to be aware of the effect of these variables and design for the worst-case scenario. This problem is extremely severe for ultra-fine talc powders.

As a matter of conventional arts, ultra-fine talc powders are usually manufactured by granulators. However, the main disadvantage of these conventional granulators is that the ultra-fine talc powders thus produced are usually of unsatisfactory quality. In recent years, granulators comprising twin-screw barrels have been developed to produce talc powders of higher quality.

A conventional twin-screw granulator usually comprises a fluidizing bed, a twin-screw assembly, and a plurality of talc powders inputting chambers communicated with the twin-screw assembly for allowing mixing of talc powders to the twin-screw assembly via the talc powders inputting chambers. The operation is as follows: the fluidizing bed is adapted for melting a predetermined amount of plastic materials at a predetermined rate, talc powders are inputted into the twin-screw assembly via the talc powder inputting chambers. The plastic materials and the talc powders are then mixed in the twin-screw assembly to form a fluid state mixture, which is then cut and condensed into a plurality of talc powders mixture for further processing or act as raw materials of a wide variety of plastic products.

A common problem for this conventional method of manufacturing the plastic raw materials is that the higher the proportion of the talc powders, the more difficult for the talc powders mixture having uniform composition and materials properties. Moreover, the smaller the talc powders, the more difficult for mixing the talc powders with the plastic materials carried by the twin-screw assembly, because the smaller the talc powders, the more air is mixed in between individual talc powders so as to make the relevant mixing process with the plastic materials more difficult.

Consequently, one may see that conventional methods and systems of manufacturing talc powders materials are ineffective, inefficient, as well as complicated in procedures. All these directly increase the cost of talc powders materials and plastic products in which these talc powders materials are mixed. As a result, conventional methods and systems for manufacturing talc powders materials substantially limit the extent of technological development in that area.

### SUMMARY OF THE PRESENT INVENTION

A main object of the present invention is to provide a system and method of manufacturing talc powder materials, wherein the talc powder materials so produced is of high quality, and optimal for use as raw materials for manufacturing other plastic products.

Another object of the present invention is to provide a system and method of manufacturing talc powder materials, which comprises a step of pre-treating a plurality of talc powders before mixing with plastic materials so as to substantially mitigate the above-mentioned problems for conventional talc powders manufacturing methods and systems.

Another object of the present invention is to provide a system and method of manufacturing talc powder materials, which are capable of substantially enhancing the manufacturing capacity of the talc powder materials as compared to conventional method and system of manufacturing the talc powder materials.

Another object of the present invention is to provide a system and method of manufacturing talc powder materials, which is capable of manufacturing talc powder materials having an enhanced proportion of talc powders as compared with convention talc powder materials, so as to substantially improve a materials performance of the talc powder materials manufactured by the method of the present invention.

Another object of the present invention is to provide a system and method of manufacturing talc powder materials, which are easy and economical to implement, so that the present invention can be utilized for most talc powder materials manufacturers.

Accordingly, in order to accomplish the above objects, the present invention provides method of manufacturing ultra-fine talc powder materials, comprising the steps of:

(a) adding a predetermined amount of pre-treatment agent with a predetermined amount of talc powders to prevent the talc powders from agglomerating with each other;

(b) performing activating treatment to the pre-treated talc powders to form activated talc powders for enhancing a compatibility of the talc powders with a predetermined plastic materials;

(c) mixing the activated talc powders with a predetermined proportion of fluidized plastic materials to form a fluidized plastic-powders mixture, wherein the fluidized plastic-powder mixture is condensed to form solid plastic-powders mixture; and

(d) hot-cutting the solid plastic-powders mixture until the solid plastic-powders mixture forms the ultra-fine talc-powders materials as raw materials for manufacturing plastic products.

Moreover, the present invention also provides an ultra-fine talc powder mixture, comprising:

a predetermined amount of talc powders;

a predetermined amount of pre-treatment agent mixing with the talc powders to form pre-treated talc powders;

a predetermined amount of activating agent mixing with the pre-treated talc powders under activating treatment to form activated talc powders for enhancing a compatibility of the talc powders with a predetermined plastic materials; and

a predetermined proportion of fluidized plastic materials mixing the activated talc powders to form a fluidized plastic-powders mixture, wherein the fluidized plastic-powder mixture is condensed to form a solid plastic-powders mixture such that the solid plastic-powders mixture is hot-cut until the solid plastic-powders mixture forms the ultra-fine talc-powder mixture as raw materials for manufacturing plastic products.

Finally, the present invention also provides an ultra-fine talc powder material manufacturing system, comprising:

a pre-treating unit which comprises a pre-treatment agent supplier providing a predetermined amount of pre-treatment agent mixing with a predetermined amount of talc powders to form pre-treated talc powders, and an activating treatment treating the pre-treated talc powders with an activating agent under activating treatment to form activated talc powders for enhancing a compatibility of the talc powders with a predetermined plastic materials; and

a talc powder machine which comprises a treatment line mixing the activated talc powders with a predetermined proportion of fluidized plastic materials to form a fluidized plastic-powders mixture, and a hot-cutter arranged in such a manner that when the fluidized plastic-powder mixture is condensed to form solid plastic-powders mixture, the solid plastic-powders mixture is hot-cut until the solid plastic-powders mixture forms the ultra-fine talc-powders materials as raw materials for manufacturing plastic products.

These and other objectives, features, and advantages of the present invention will become apparent from the following detailed description, the accompanying drawings, and the appended claims.

### BRIEF DESCRIPTION OF THE DRAWINGS

FIG. 1 is a method of manufacturing ultra-fine talc powder materials according to a preferred embodiment of the present invention.

FIG. 2 is a schematic diagram of an ultra-fine talc powder material manufacturing system according to the above preferred embodiment of the present invention.

### DETAILED DESCRIPTION OF THE PREFERRED EMBODIMENT

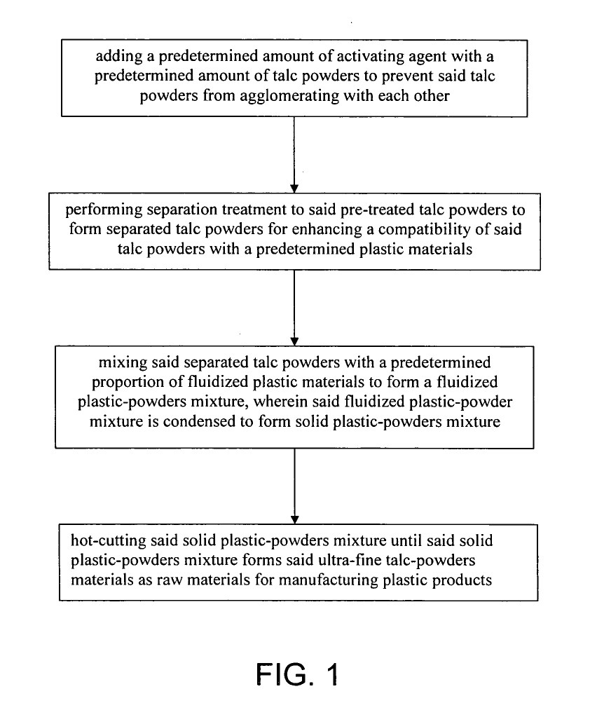

Referring to FIG. 1 of the drawings, a method of manufacturing ultra-fine talc powder materials is illustrated, in which the method comprises the steps of:

(a) pre-treating a predetermined amount of talc powders for preventing the talc powders from agglomerating with each other;

(b) performing an separation treatment to the pre-treated talc powders to form separated talc powders for enhancing a compatibility of the talc powders with a predetermined plastic materials;

(c) mixing the separated talc powders with a predetermined proportion of fluidized plastic materials to form a fluidized plastic-powders mixture, wherein the fluidized plastic-powder mixture is condensed to form solid plastic-powders mixture; and

(d) hot-cutting the solid plastic-powders mixture until the solid plastic-powders mixture forms the ultra-fine talc powders materials as raw materials for manufacturing plastic products.

According to the preferred embodiment of the present invention, step (a) comprises the steps of:

(a.1) adding a predetermined amount of activating agent to a predetermined amount of talc powders; and

(a.2) blending the predetermined amount of talc powders with the activating agent by a high-speed blending device so as to mix the talc powders with the activating agent and substantially prevent each of the talc powders from agglomerating with each other.

It is worth mentioning that the activating agent is capable of enhancing a mobility of each of the talc powders so as to enhance its compatibility with the plastic materials. The activating agent is preferably embodied as steraic acid, silane coupling agent, or aluminate ester coupling agent.

In step (a.2), the blending process is accomplished by a high-speed blending machine comprising a plurality of blending blades which are driven to rotate at about 1700 revolutions per minute for substantially separating each of the talc powders in order to prevent the talc powders from agglomerating with the addition of the separation agent. It is worth mentioning that once the talc powders are substantially blended as stated in step (a.2), the compatibility of the talc powders and the plastic materials will be substantially enhanced.

Step (b) comprises the steps of:

(b.1) adding a predetermined amount of separation agent to the pre-treated talc powders for substantially mixing the separation agent with the pre-treated talc powders so as to embed each of the talc powders by the separation agent.

There are two objectives for this step. First, by separating the talc powders with the separation agent, the talc powders will be more compatible with the plastic materials for producing high quality talc powders materials. Second, by embedding each of the talc powders with the separation agent, there will be less air (or even no air) occupying in between each of the talc powders so as to render easier the mixing process between the talc powders and the plastic materials. According to the preferred embodiment of the present invention, the separation agent is PE wax, PP wax, polystyrene oligmer, or random PP.

In step (c), the mixing of the talc powders with the plastic materials is preferably performed by a talc powder machine. A predetermined amount of pre-treated and separated talc powders are mixed with fluidized plastic materials for forming the talc powders raw materials. Since the talc powders are pre-treated and substantially separated, their compatibility with the plastic materials is thereby maximized.

According to the preferred embodiment of the present invention, the method of manufacturing talc powders materials further comprises the steps of:

(1) adding a predetermined amount of POE to the mixture of the plastic materials and the talc powders; and

(2) adding a predetermined amount of linear low density polyethylene (LLDPE) to the mixture of the plastic materials and the talc powders; and

(3) adding a predetermined amount of lubricating agent to the mixture of plastic materials and the talc powders.

It is worth mentioning that step (1) to step (3) are actually optional so that one may perform all these steps to produce the talc powders materials or any one of them. However, for the optimal performance of the talc powders materials, it is advised that step (1) to step (3) are performed.

From the above description, it can be shown that the present invention also provides a ultra-fine talc powder mixture, comprising:

a predetermined amount of talc powders;

a predetermined amount of activating agent mixing with the talc powders to form pre-treated talc powders;

a predetermined amount of separation agent mixing with the pre-treated talc powders under separation treatment to form separated talc powders for enhancing a compatibility of the talc powders with a predetermined plastic materials; and

a predetermined proportion of fluidized plastic materials mixing the activated talc powders to form a fluidized plastic-powders mixture, wherein the fluidized plastic-powder mixture is condensed to form a solid plastic-powders mixture such that the solid plastic-powders mixture is hot-cut until the solid plastic-powders mixture forms the ultra-fine talc-powder mixture as raw materials for manufacturing other plastic products.

The ultra-fine talc powders mixture further comprises a predetermined amount of POE, a predetermined amount of linear low density polyethylene (LLDPE) and a predetermined amount of lubricating agent which are added to the mixture of talc powders and plastic materials for achieving optimal performance of the present invention.

According to the preferred embodiment of the present invention, the preferred percentage of each of the constituents is listed as follows:

| | |
|---|---|
| (a) talc powders | 70% |
| (b) activating agent | 2% |
| (c) separation agent | 8% |
| (d) POE | 6% |
| (e) LLDPE | 14% |

Alternatively, the composition of the talc powders mixture is as follows:

| | |
|---|---|
| (a) talc powders | 80% |
| (b) activating agent | 2% |
| (c) separation agent | 8% |
| (d) lubricating agent | 3% |
| (e) PP | 7.5% |

Moreover, the composition of the talc powders mixtures may be embodied as follows:

| | |
|---|---|
| (a) talc powders | 70% |
| (b) activating agent | 1% |
| (c) separation agent | 8% |
| (d) lubricating agent | 2% |

From the above date, one may appreciate that the result talc-powders materials can contain up to 80% of talc powders—a percentage which is substantially higher than that of convention talc-powder materials. It is important to point out that, however, that the present invention does not suffer from the problems encountered by conventional methods of manufacturing talc-powder materials.

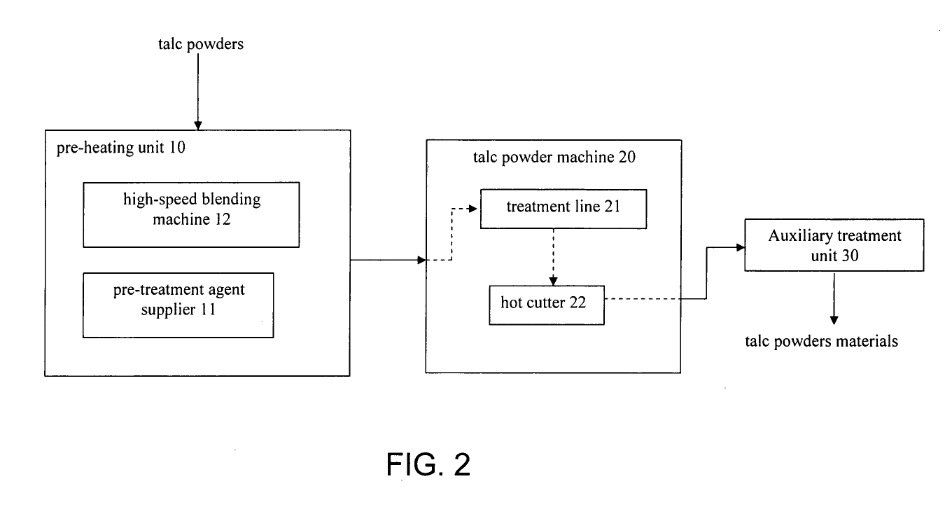

Referring to FIG. 2 of the drawings, an ultra-fine talc powder material manufacturing system according to the preferred embodiment of the present invention is illustrated, in which the ultra-fine talc powder material manufacturing system comprises a pre-treating unit 10, and a talc powder machine 20.

The pre-treating unit 10 comprises a pre-treatment agent supplier 11 providing a predetermined amount of activating agent mixing with a predetermined amount of talc powders to form pre-treated talc powders, and an separation treatment unit 12 treating the pre-treated talc powders with an separation agent under separation treatment to form separated talc powders for enhancing a compatibility of the talc powders with a predetermined plastic materials.

The talc powder machine 20 comprises a treatment line 21 mixing the activated talc powders with a predetermined proportion of fluidized plastic materials to form a fluidized plastic-powders mixture, and a hot-cutter 22 arranged in such a manner that when the fluidized plastic-powder mixture is condensed to form solid plastic-powders mixture, the solid plastic-powders mixture is hot-cut until the solid plastic-powders mixture forms the ultra-fine talc-powders materials as raw materials for manufacturing plastic products.

The talc powder machine 20 is preferably embodied as a twin-screw talc powder materials manufacturing machine wherein the talc powders mixture is hot-cut to form ultra-fine talc powders materials.

As a result, the pre-treating unit 10 comprises a high-speed blending machine 12 comprising a plurality of blending blades which are driven to rotate at around 1700 revolutions per minute for substantially blending said talc powders so as to substantially separate each of the talc powders for producing the talc powders materials in high quality.

The ultra-fine talc powder material manufacturing system further comprises an auxiliary treatment unit 30 operatively communicated with the talc powder machine and adapted for mixing a plurality auxiliary additives to the fluidized powder-plastic mixture so as to further enhance a quality of the talc powders materials.

The auxiliary additives are corresponding with the optional steps as mentioned above, viz. POE, linear low density polyethylene (LLDPE), and/or lubricating agent.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of manufacturing ultra-fine talc powder materials, comprising the steps of:
(a) adding a predetermined amount of activating agent with a predetermined amount of talc powders to prevent said talc powders from agglomerating with each other;
(b) performing separation treatment to said pre-treated talc powders to form separated talc powders for enhancing a compatibility of said talc powders with a predetermined plastic materials;
(c) mixing said separated talc powders with a predetermined proportion of fluidized plastic materials to form a fluidized plastic-powders mixture, wherein said fluidized plastic-powder mixture is condensed to form solid plastic-powders mixture; and
(d) hot-cutting said solid plastic-powders mixture until said solid plastic-powders mixture forms said ultra-fine talc-powders materials as raw materials for manufacturing plastic products.

2. The method, as recited in claim 1, wherein said step (a) comprises the steps of:
(a.1) adding a predetermined amount of activating agent to a predetermined amount of talc powders; and
(a.2) blending the predetermined amount of talc powders with the activating agent by a high-speed blending device so as to mix the talc powders with the activating agent and substantially prevent each of the talc powders from agglomerating with each other.

3. The method, as recited in claim 2, wherein said step (b) comprises a step of adding a predetermined amount of separation agent to said pre-treated talc powders for substantially mixing said separation agent with said pre-treated talc powders so as to embed each of said pre-treated talc powders by said separation agent for minimizing air trapped in between each of said talc powders.

4. The method, as recited in claim 2, wherein said activating agent is one of the followings: steraic acid, silane coupling agent, and aluminate ester coupling agent.

5. The method, as recited in claim 3, wherein said activating agent is one of the followings: steraic acid, silane coupling agent, and aluminate ester coupling agent.

6. The method, as recited in claim 4, wherein said step (a.2) is accomplished by a high-speed blending machine comprising a plurality of blending blades which are driven to rotate at around 1700 revolutions per minute for substantially separating each of the talc powders.

7. The method, as recited in claim 5, wherein said step (a.2) is accomplished by a high-speed blending machine comprising a plurality of blending blades which are driven to rotate at around 1700 revolutions per minute for substantially separating each of the talc powders.

8. The method, as recited in claim 6, further comprising a step of adding a predetermined amount of POE to said fluidized plastic-powder mixture.

9. The method, as recited in claim 7, further comprising a step of adding a predetermined amount of POE to said fluidized plastic-powder mixture.

10. The method, as recited in claim 8, further comprising a step of adding a predetermined amount of linear low density polyethylene (LLDPE) to said fluidized plastic-powder mixture.

11. The method, as recited in claim 9, further comprising a step of adding a predetermined amount of linear low density polyethylene (LLDPE) to said fluidized plastic-powder mixture.

12. The method, as recited in claim 10, further comprising a step of adding a predetermined amount of lubricating agent to said fluidized plastic-powder mixture.

13. The method, as recited in claim 11, further comprising a step of adding a predetermined amount of lubricating agent to said fluidized plastic-powder mixture.

14. A ultra-fine talc powder mixture, comprising:
a predetermined amount of talc powders;
a predetermined amount of activating agent mixing with said talc powders to form pre-treated talc powders;
a predetermined amount of separation agent mixing with said pre-treated talc powders under separation treatment to form activated talc powders for enhancing a compatibility of said talc powders with a predetermined plastic materials; and
a predetermined proportion of fluidized plastic materials mixing said separated talc powders to form a fluidized plastic-powders mixture, wherein said fluidized plastic-powder mixture is condensed to form a solid plastic-powders mixture such that said solid plastic-powders mixture is hot-cut until said solid plastic-powders mixture forms said ultra-fine talc-powder mixture as raw materials for manufacturing plastic products.

15. The ultra-fine talc powders mixture, as recited in claim 14, further comprising a predetermined amount of POE.

16. The ultra-fine talc powders mixture, as recited in claim 15, further comprising a predetermined amount of linear low density polyethylene (LLDPE).

17. The ultra-fine talc powders mixture, as recited in claim 16, further comprising a predetermined amount of lubricating agent.

18. An ultra-fine talc powder material manufacturing system, comprising:
a pre-treating unit which comprises an activating agent supplier providing a predetermined amount of activating agent mixing with a predetermined amount of talc powders to form pre-treated talc powders, and an separation treatment treating said pre-treated talc powders with an separation agent under activating treatment to form separated talc powders for enhancing a compatibility of said talc powders with a predetermined plastic materials; and
a talc powder machine which comprises a treatment line mixing said activated talc powders with a predetermined proportion of fluidized plastic materials to form a fluidized plastic-powders mixture, and a hot-cutter arranged in such a manner that when said fluidized plastic-powder mixture is condensed to form solid plastic-powders mixture, said solid plastic-powders mixture is hot-cut until said solid plastic-powders mixture forms said ultra-fine talc-powders materials as raw materials for manufacturing plastic products.

19. The ultra-fine talc powder material manufacturing system, as recited in claim 18, wherein said pre-treating unit comprises a high-speed blending machine comprising a plurality of blending blades which are driven to rotate at around 1700 revolutions per minute for substantially blending said talc powders so as to substantially separate each of the talc powders for producing said talc powders materials in a high quality.

20. The ultra-fine talc powder material manufacturing system, as recited in claim 19, further comprising an auxiliary treatment unit operatively communicated with said talc powder machine and adapted for mixing a plurality auxiliary additives to said fluidized powder-plastic mixture so as to further enhance a quality of said talc powders materials.
